(19) United States
(12) Patent Application Publication
(10) Pub. No.: US 2024/0138683 A1
(43) Pub. Date: May 2, 2024

(54) System and Method for Thermal Screening

(71) Applicant: C2RO Cloud Robotics Inc., Montreal (CA)

(72) Inventors: Riccardo Badalone, St. Lazare (CA); Amir Abbas Haji Abolhassani, Dollard-des-Ormeaux (CA); Aldo Enrique Vargas Moreno, Montreal (CA); Felix-Olivier Duguay, Greenfield Park (CA); Soodeh Farokhi, Verdun (CA); François Magnan, Montreal (CA); Mostafa Erfani, Brossard (CA)

(21) Appl. No.: 18/281,098

(22) PCT Filed: Mar. 9, 2022
(86) PCT No.: PCT/CA2022/050339

§ 371 Date: Sep. 8, 2023

Related U.S. Application Data
(60) Provisional application No. 63/158,585, filed on Mar. 9, 2021.

(51) Int. Cl. A61B 5/01; A61B 5/00; G06T 7/00; G06T 7/20; G06T 7/50; G06V 10/143; G06V 10/25; G06V 40/10; G06V 40/16; G16H 50/30; H04N 23/11; H04N 23/13

(52) U.S. Cl. CPC A61B 5/01; A61B 5/7485; G06T 7/0012; G06T 7/20; G06T 7/50; G06V 10/143; G06V 10/25; G06V 40/103; G06V 40/165; G06V 40/171; G06V 40/178; G16H 50/30; H04N 23/11; H04N 23/13; G06T 2207/10048; G06T 2207/30201

(57) ABSTRACT

A method for performing thermal screening is described. The method includes capturing at least one non-thermal image of a scene having a person; capturing at least one thermal image of the scene having the person; determining a region of interest of the non-thermal image; determining, based on a body sub-region of the non-thermal image corresponding to the body of the person within the scene, a distance value representative of a physical distance of the person from the thermal camera; determining a region of interest of the thermal image corresponding to the region of interest of the non-thermal image; determining a raw temperature value for the person based on the region of interest of the thermal image; and normalizing the raw temperature value based on at least the distance value to determine a normalized temperature value. A corresponding system and non-transitory computer-readable medium are also provided.

### RELATED PATENT APPLICATION

This application claims the benefit of and priority to U.S. Provisional Patent Application No. 63/158,585, entitled SYSTEM AND METHOD FOR THERMAL SCREENING and filed Mar. 9, 2021, the entirety of which is incorporated herein by reference.

### TECHNICAL FIELD

The present disclosure generally relates to a system and method for thermal screening within a space where one or more people are present, and more particularly for automated thermal screening using captured non-thermal image(s) and thermal image(s).

### BACKGROUND

Thermal screening of people in a space is becoming an increasingly relevant application for ensuring public health. Thermal screening seeks to evaluate the temperature of a person. Since the skin temperature of a person is indicative of a presence of a possible health condition in that person, screening the person's skin temperature can be an effective way of detecting whether that person has a possible health condition. When a person is identified through thermal screening as having a possible health condition, additional actions may be taken, such as further evaluating the person or requesting that the person withdraws from contact with other people or objects.

### SUMMARY

According to one aspect, there is provided a method for performing automated thermal screening. The method includes capturing at least one non-thermal image of a scene having a person within the scene, capturing at least one thermal image of the scene having the person within the scene, determining a region of interest of the non-thermal image, determining, based on a body sub-region of the non-thermal image corresponding to the body of the person within the scene, a distance value representative of a physical distance of the person from the thermal camera, determining a region of interest of the thermal image corresponding to the region of interest of the non-thermal image, determining a raw temperature value for the person based on the region of interest of the thermal image, and normalizing the raw temperature value based on at least the distance value to determine a normalized temperature value.

According to another aspect, there is provided a method for synchronizing a series of non-thermal images captured of a scene with a series of thermal images captured of the scene. The method includes for a given non-thermal image of the series of non-thermal images, detecting, a non-thermal coarse sub-region corresponding to a person captured within the scene, for the given non-thermal image, identifying a subset of the thermal images taken within a temporal neighborhood of the given non-thermal image, for each thermal image of the subset, detecting a respective thermal coarse sub-region corresponding to the person within the scene, identifying, from the subset of the thermal images, a given thermal image having its thermal coarse sub-region presenting a sufficient correspondence with the coarse sub-region of the given non-thermal image, the given thermal image being selected as a synchronized thermal image corresponding to the given non-thermal image.

According to yet another aspect, there is provided a system for performing privacy-aware movement tracking. The system includes at least one processor and at least one memory coupled to the processor and storing instructions executable by the processor and that such execution causes the processor perform operations including: capturing at least one non-thermal image of a scene having a person within the scene, capturing at least one thermal image of the scene having the person within the scene, determining a region of interest of the non-thermal image, determining, based on a body sub-region of the non-thermal image corresponding to the body of the person within the scene, a distance value representative of a physical distance of the person from the thermal camera, determining a region of interest of the thermal image corresponding to the region of interest of the non-thermal image, determining a raw temperature value for the person based on the region of interest of the thermal image, and normalizing the raw temperature value based on at least the distance value to determine a normalized temperature value.

According to yet another aspect, there is provided a system for synchronizing a series of non-thermal images captured of a scene with a series of thermal images captured of the scene. The system includes at least one processor and at least one memory coupled to the processor and storing instructions executable by the processor and that such execution causes the processor to perform operations including: for a given non-thermal image of the series of non-thermal images, detecting, a non-thermal coarse sub-region corresponding to a person captured within the scene, for the given non-thermal image, identifying a subset of the thermal images taken within a temporal neighborhood of the given non-thermal image, for each thermal image of the subset, detecting a respective thermal coarse sub-region corresponding to the person within the scene, and identifying, from the subset of the thermal images, a given thermal image having its thermal coarse sub-region presenting a sufficient correspondence with the coarse sub-region of the given non-thermal image, the given thermal image being selected as a synchronized thermal image corresponding to the given non-thermal image.

### BRIEF DESCRIPTION OF THE DRAWINGS

For a better understanding of the embodiments described herein and to show more clearly how they may be carried into effect, reference will now be made, by way of example only, to the accompanying drawings which show at least one exemplary embodiment, and in which:

FIG. 1 illustrates a schematic diagram of a system for performing thermal screening according to one example embodiment;

FIG. 2 is a schematic diagram showing a deployment of the thermal screening system according to one example embodiment;

FIG. 3 is a schematic diagram illustrating representative images processing steps applied as part of the thermal screening process according to one example embodiment;

FIG. 4 is a schematic diagram illustrating detailed modules and operations within the thermal screening system according to one example embodiment;

FIG. 5 is a flowchart illustrating the operational steps of a method for performing thermal screening according to one example embodiment;

FIG. 6a is a schematic diagram illustrating an experimental setup in a first calibration phase according to one example embodiment;

FIG. 6b is a schematic diagram illustrating the experimental setup in a second calibration phase according to one example embodiment;

FIG. 6c is a schematic diagram illustrating an operational setup of the thermal screening system according to one example embodiment;

FIG. 7 is a graph showing the performance of the thermal screening according to an experimental setup in comparison to a commercially available thermal screening system;

FIG. 8 shows hardware components for implementing a thermal screening system according to one example embodiment;

FIG. 9 illustrates a use case for the thermal screening system according to one example embodiment;

It will be appreciated that for simplicity and clarity of illustration, elements shown in the figures have not necessarily been drawn to scale. For example, the dimensions of some of the elements may be exaggerated relative to other elements for clarity.

### DETAILED DESCRIPTION

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art, that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

It was observed that many available systems for performing thermal screening require strict operating conditions in order to ensure accuracy and/or effectiveness. Such strict operating conditions can include one or more of:
requiring the human test subject to be located at a precise position (ex: distance, height, angle) relative to thermal screening equipment;
requiring the human test subject to take a participatory action, such as moving to the line position or taking off accessories (ex: glasses), which may improve the accuracy of the thermal screening;
requiring a narrow range of ambient temperature;
the thermal screening is active, in that it is readily apparent to the person that they are being screened;
requiring frequent calibration to make sure the measurement stays accurate and it is drifting.

Such drawbacks of available thermal screening systems cause users to find thermal screening to be burdensome, intrusive, and costly, which decreases the adoption and effectiveness of such thermal screening systems.

The thermal screening described herein according to various example embodiments may be implemented as methods, apparatus, systems, computing devices, computing entities, and/or the like. As such, embodiments may take the form of an apparatus, system, computing device, computing entity, and/or the like executing instructions stored on a computer-readable storage medium to perform certain steps or operations. However, embodiments of the present invention may also take the form of an entire hardware embodiment performing certain steps or operations. Such devices can each comprise at least one processor, a data storage system (including volatile and non-volatile memory and/or storage elements). For example, and without limitation, the programmable computer may be a programmable logic unit, a mainframe computer, server, personal computer, cloud-based program or system, laptop, personal data assistant, cellular telephone, smartphone, wearable device, tablet device, virtual reality devices, smart display devices (ex: Smart TVs), set-top box, video game console, or portable video game devices.

Embodiments of the present are described below with reference to block diagrams and flowchart illustrations. Thus, it should be understood that each block of the block diagrams and flowchart illustrations, respectively, may be implemented in the form of a computer program product, an entirely hardware embodiment, a combination of hardware and computer program products, and/or apparatus, systems, computing devices, computing entities, and/or the like carrying out instructions on a computer-readable storage medium for execution. Such embodiments can produce specifically-configured machines performing the steps or operations specified in the block diagrams and flowchart illustrations. Accordingly, the block diagrams and flowchart illustrations support various combinations of embodiments for performing the specified steps or operations.

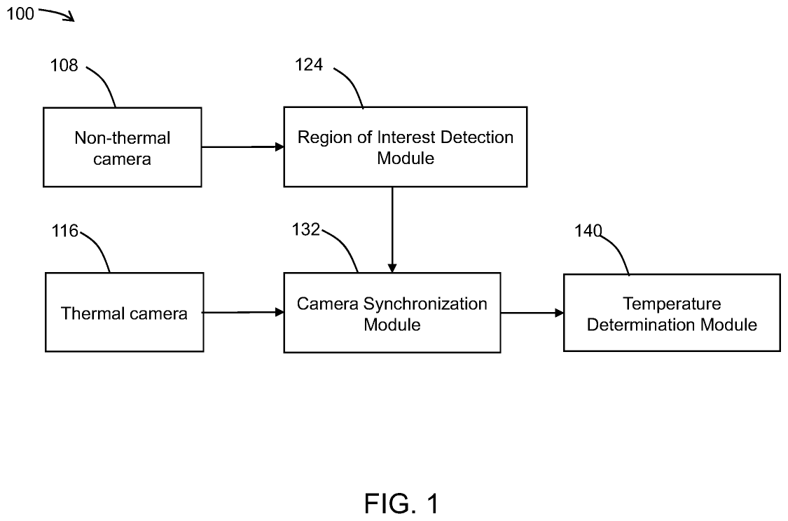

Referring now to FIG. 1, therein illustrated is a schematic diagram of a system 100 for performing thermal screening according to one example embodiment. The thermal screening system 100 is configured to receive at least one non-thermal image of a scene captured by a non-thermal camera 108. The non-thermal camera 108 refers to any type of camera that captures signals other than ones that are representative of temperature information. The non-thermal camera 108 can refer to a camera that captures signals in the visible light range, which are often called RGB cameras. The non-thermal camera 108 can be a standard digital camera, such as an IP surveillance camera.

The thermal screening system 100 is also configured to receive at least one thermal image of the scene captured by a thermal camera 116. The thermal camera 116 refers to any type of camera that captures signals that are representative of temperature information emitted by blackbodies found within the captured scene. The thermal camera 116 can capture signals having wavelengths that are longer than wavelengths in the visible light range. The thermal camera 116 can be an infrared camera sensitive to wavelengths in the infrared range.

The at least one captured non-thermal image and the at least one captured thermal image have a sufficient temporal and spatial correspondence, in that the visual information of the non-thermal image is relevant in time and position to the thermal information of the thermal image. In other words, the non-thermal image and the thermal image are captured sufficiently close in time and capture substantially the same scene of a real-world space. As described elsewhere herein, a synchronizing process may be carried out to temporally synchronize (i.e. in time) one or more thermal images captured by the thermal camera 116 with one or more non-thermal images captured by the thermal camera 108.

The thermal screening system 100 includes a region of interest detection module 124 configured for determining a region of interest of the non-thermal image. As described elsewhere herein, the region of interest detection module 124 applies a plurality of image processing steps to identify the region of interest within the non-thermal image.

The thermal screening system 100 also includes a synchronization module 132 configured for determining a region of interest of the thermal image corresponding to the determined region of interest of the non-thermal image. As described elsewhere herein, the determining of the region of interest of the thermal image may include ensuring synchronization of non-thermal images captured by, and received from, the non-thermal camera 108 with thermal images captured by, and received from, the thermal camera 116. The synchronization ensures that the appropriate thermal image is paired with the non-thermal image when determining the region of interest of the thermal image.

The synchronization also ensures an appropriate matching of the region of interest of the non-thermal image to the region of the thermal image. As described elsewhere herein, when deployed, the non-thermal camera 108 may be offset positionally and/or orientationally relative to the thermal camera 116. Accordingly, coordinates of a given pixel in the non-thermal image associated with a real-life spatial position within a scene are transformed according to the thermal camera/non-thermal camera spatial offset in order to determine the coordinates of a corresponding pixel within the thermal image that is associated to the same real-life spatial position within the scene. For example, an affine transformation can be applied to translate coordinates of the non-thermal image to coordinates of the thermal image, and vice versa. The amount and degree of the transformation can be determined in a calibration phase and becomes intrinsic and invariant to the non-thermal camera 108 and thermal camera pairing 116. That is, the same transformation is applied throughout the operation until the offset of the cameras is altered and another calibration becomes necessary.

The thermal screening system 100 also includes a temperature determination module 140 configured for determining a temperature value based on the region of interest of the thermal image. The temperature value is determined from pixel values within the region of interest of the thermal image, the pixel values being representative of the temperature of corresponding portions of the scene captured by the thermal image. For example, for a 12-bit thermal image, the temperature value can be obtained by taking the pixel values from 0 to 65,000.

It will be understood that the thermal screening system 100 can be delivered without the non-thermal camera 108 and the thermal camera 116. For example, the thermal screening system 100 can be combined with existing non-thermal camera 108 and thermal camera 116 that have already been installed.

Alternatively, the thermal screening system 100 can include the non-thermal camera 108 and the thermal camera 116, whereby modules of the system 100 are configured according to specifications of the non-thermal camera 108 and the thermal camera 116.

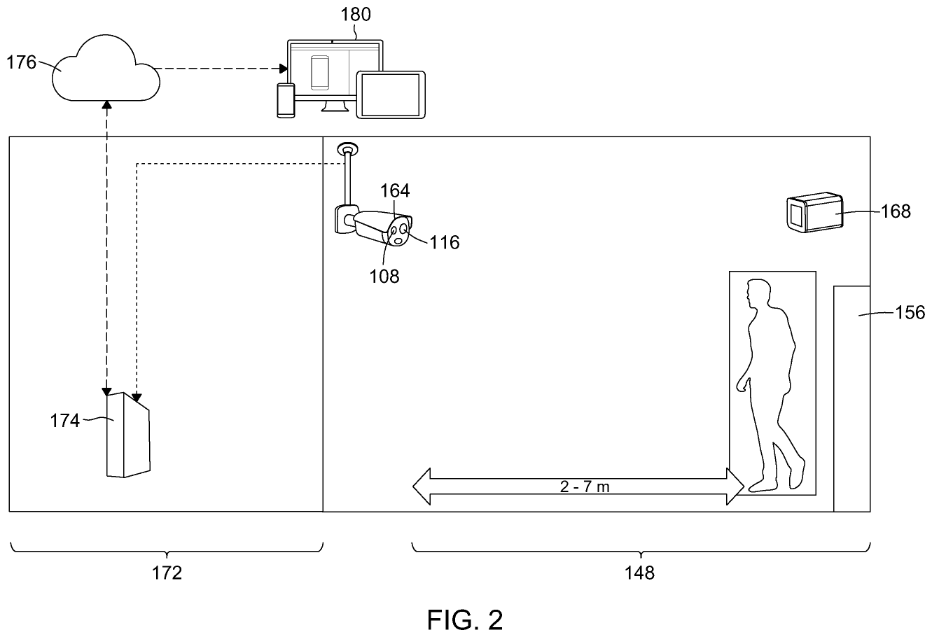

Referring now to FIG. 2, therein illustrated is a schematic diagram showing a deployment of the thermal screening system 100 with the thermal camera 108 and the non-thermal camera 116 according to one example embodiment.

The non-thermal camera 108 is installed and appropriately oriented to be effective to capture non-thermal images of a scene 148. Similarly, the thermal camera 116 is installed and oriented to be effective to capture respective thermal images of the scene 148. In the example illustrated in FIG. 2, the non-thermal camera 108 and the thermal camera 116 are illustrated as being integrated together within a hybrid camera system 164. However, it will be understood that the non-thermal camera 108 and the thermal camera 116 may be implemented as separate devices in other example embodiments.

The scene can cover a floor area that will receive foot traffic for which thermal screening is desired. In the non-limiting example illustrated in FIG. 2, the floor area corresponding to the scene 148 covered by the non-thermal camera 108 is an area in the vicinity of an entrance door 156. A person, being a potential test subject, is illustrated being present in the scene 148.

A reference blackbody emitter 168 may also be located within the scene being captured by the thermal camera 116. The reference blackbody emitter 168 serves as a reference, which is used to more precisely measure temperature values of objects in the scene captured by the thermal camera 116.

According to the example embodiment illustrated in FIG. 2, the thermal screening system 100 is implemented as a combination of a processing component 172 (which may be remote and/or local) and one or more user devices 180. The non-thermal image(s) and thermal image(s) can be received at the processing component 172 and the processing of the image(s) is carried out according to the functionalities of the thermal screening system 100 described herein. The processing further determines a screening-related alert, which is sent to the one or more user devices 180, so that a human operator can evaluate the alert and take any appropriate further action as part of an overall thermal screening process.

As further illustrated in the example embodiment, the processing component 172 can include an edge server 174 (located at the local network of the cameras 108 and 116) and a cloud network component 176. Processing tasks can be split between the edge server 174 and the cloud network component 176. For example, the cloud network component 176 can manage alerts and monitoring data that are to be sent to the user devices 180. Having the cloud component can help for scalability purposes, such as serving multiple monitored sites (ex: for the same customer) and aggregating analytics data (ex: to understand data patterns over the multiple monitored sites). However, it will be understood that the processing component 172 can be implemented without the cloud network component 176.

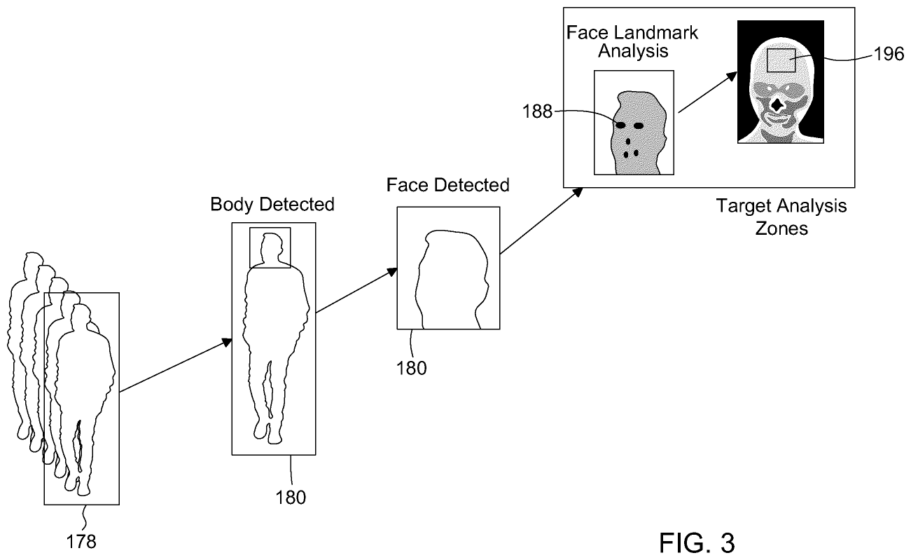

Referring now to FIG. 3, therein is a schematic diagram illustrating representative image processing steps applied as part of the thermal screening process performed by thermal screening system 100, according to one example embodiment.

The non-thermal camera 108 and the thermal camera 116 are configured when deployed so that both cameras 108, 116 are able to capture images of the full body of a person passing through the scene 148 being monitored by the cameras 108, 116.

According to one example embodiment, capturing the full body of the person includes capturing at least a face of the person as well as at least a portion of the lower body of the person.

According to some example embodiments, and as illustrated in FIG. 3, capturing the full body of the person includes capturing the face of the person all the way down to the feet of the person.

According to some example embodiments, and as illustrated in FIG. 3, the region of interest detection module 124 is operable to apply a body detection algorithm to detect, using a body detection algorithm, a body sub-region of the non-thermal image corresponding to the full body of the person located within the scene captured by the non-thermal camera 108. According to the example illustrated in FIG. 3, the body sub-region of a first detected person is identified by a first bounding box 178.

Continuing with FIG. 3, the region of interest detection module 124 is further operable to apply a face detection algorithm to detect, within the body sub-region, a face sub-region of the non-thermal image corresponding to the face of the person. According to the example illustrated in FIG. 3, the face sub-region of the first detected person is identified by a second bounding box 180.

The region of interest detection module 124 is further operable to determine a region of interest within the non-thermal image captured by the non-thermal camera 108. The region of interest can correspond to the detected face sub-region or to a portion of the detected face sub-region.

According to one example embodiment, the region of interest of the non-thermal image is defined by the position of facial landmarks within the face sub-region of the non-thermal image. Accordingly, determining the region of interest of the non-thermal image proceeds by first detecting one or more facial features within the face sub-region that act as landmarks. Detected facial landmarks can include one or more of an eyes landmarks, a nose landmark, and mouth landmark(s) (ex:

represented by corners of the mouth). Visual markers 188 are shown in FIG. 3 to identify the detected facial landmarks.

It will be understood that the body detection, face detection and facial landmark detection carried out by the region of interest detection module 124 are applied to the non-thermal image captured by the non-thermal image camera 108.

Once the region of interest of the non-thermal image (ex: as defined by the position facial landmarks) has been determined and defined as the region of interest of the non-thermal image, the coordinates of the region of interest the non-thermal image are transmitted to the camera synchronization module 132.

The synchronization module 132 identifies the thermal image corresponding in time to the non-thermal image that was treated and for which the non-thermal region of interest was detected. The synchronization module 132 determines the coordinates of the region of interest within the thermal corresponding to (or relative to) the coordinates of the region of interest of the non-thermal image. This determination can include applying a transformation to the coordinates of the region of interest of the non-thermal image to calculate the corresponding coordinates within the thermal image, such as being applying an affine transformation. As mentioned elsewhere, the transformation is effective to correct for the positional and orientational offset between the positions and orientations of non-thermal camera 108 and the thermal image camera 116, which is also known as spatial stereo transformation.

Determining the coordinates of the region of interest within thermal image corresponding to the coordinates of the non-thermal image may further include applying a positional adjustment so that the region of interest of thermal image represents a real-world location on the person that is a portion of, or is different from, the real-world location on the person represented by the region of interest of the non-thermal image.

According to one example implementation, the region of interest of the thermal image is determined so that it corresponds to the forehead region on the person. It will be appreciated that this region is located slightly above the facial landmarks (ex: eyes, nose, mouth) of the person. Accordingly, the positional adjustment is applied to determine the region of interest of the thermal image relative to the coordinates of the region of interest of the non-thermal image taking into account the difference in location between the facial landmarks and the forehead region.

According to another example implementation, the region of interest of the thermal image is determined so that it corresponds to a region of the person relative to the facial landmarks (ex: eyes, nose, mouth), such as within the boundaries of a face bounding box determined from the facial landmarks.

According to yet another example implementation, the region of the interest on the image is determined so that it corresponds to a region of the face of the person other than the forehead region.

Continuing with FIG. 3, the region of interest of the thermal image determined by the synchronization module 132 is identified by the bounding box 196.

It will be appreciated that the bounding box 196 is located over the forehead of the human subject within the thermal image.

The coordinates of the region of interest of the thermal image, as determined by the camera synchronization module 132, are received by the temperature determination module 140. The temperature determination module 140 determines the measured temperature value based on pixel values within the region of interest of the thermal image, the pixel values being representative of the temperature of corresponding portions of the scene captured by the thermal image. As described elsewhere herein, the temperature value determined from the pixel values of the region of interest, without further treatment, represents a raw measured temperature value. This raw measured temperature value can be further normalized based on one or more normalizing factors. The normalizing translates the raw temperature value to a representative core body temperature of the person captured in the thermal image.

While the preceding section describing image processing steps being applied to a single non-thermal image and a single corresponding thermal image, it will be understood that the temperature value can be determined from a plurality of images (non-thermal images and thermal images) captured for a single person located within the scene. The determination of the plurality of images can provide a more accurate temperature value, such as, by correcting for any spurious readings that may be found in a single thermal image.

According to example embodiments in which the temperature value is determined from a plurality of images, the non-thermal image region of interest detection module 124 receives a series of a plurality of non-thermal images of the scene captured by the non-thermal camera 108, the plurality of images of the scene having the person subject. The series of images can form a video captured by the non-thermal camera 108. Similarly, the camera synchronization module 132 receives a corresponding series of thermal images of the same scene captured by the thermal camera 116, these images also having the person subject. Each thermal image of the series can correspond to a respective one of the series of the plurality of non-thermal images.

The synchronization module 132 is operable to receive the region of interest for each of the non-thermal images. The synchronization module 132 also receives a series of thermal images. For each of the non-thermal images, a corresponding thermal image is identified. Then for each of the plurality of thermal images, a respective region of interest is identified based on the region of interest of the corresponding non-thermal image.

The temperature determination module 140 further determines a respective intermediate temperature value for the region of interest for each of the thermal images. Accordingly, a series of intermediate measured temperature values are determined.

According to one example embodiment, a single measured temperature value for the whole series of images (thermal and non-thermal images) is determined by calculating an average of the series of intermediate temperature values determined from the region of interest of each of the thermal images. The average temperature value can be an average raw temperature value, which is subsequently normalized. Alternatively, the average temperature value can be an average of the respective normalized temperature value determined for each frame of the series of images.

According to various example embodiments, the synchronization module 132 is operable to temporally (i.e. in time) synchronize the series of non-thermal images with the series of thermal images. When so synchronized, each of the non-thermal images can be synced with a corresponding thermal image.

Synchronizing the series of non-thermal images with the series of thermal images includes detecting, for a given non-thermal image of the series of non-thermal images, a coarse sub-region corresponding to the person within the scene. The given non-thermal image can be one image in a sub-series of non-thermal images that capture the person within the scene. The given non-thermal image can be the first image in this sub-series. The coarse sub-region can be any sub-region that is less precisely defined than the region of interest of the non-thermal image. For example, the coarse sub-region can be the body sub-region corresponding to the full body of the person, or a portion of the body sub-region. The coarse sub-region can be the face sub-region. In either case, the coarse sub-region is less precise than the facial landmarks defining the region of interest of the non-thermal image.

Synchronizing the series of non-thermal images with the series of thermal images further includes, identifying, for the given non-thermal image, a subset of thermal images captured within a temporal neighborhood of the given non-thermal image. The subset of thermal images falling within the temporal neighborhood are thermal images captured within a time range beginning slightly before the time of capture of the given non-thermal image and ending slightly after the time of capture of the given non-thermal image. The width of the time range is appropriately selected so that there is a sufficient relevance (i.e. capturing the same person at the same location within the scene) between the given non-thermal image and at least one of thermal images within the subset of thermal images.

For each thermal image of the subset of thermal images, a respective thermal coarse sub-region corresponding to the person within the scene is detected. The coarse sub-region can be any sub-region that is less precisely defined than the region of interest of the thermal image that is ultimately determined. For example, the coarse sub-region can be a body sub-region corresponding to the person, or a portion of the body sub-region. The coarse sub-region can be the face sub-region. In either case, the coarse sub-region of the thermal image is less precise than the region of interest of the non-thermal image.

According to one example embodiment, the coarse sub-region of each thermal image is detected by identifying a cluster of adjacent pixels in the thermal image having raw temperature values that are close in value to an expected temperature value of a human face. A cluster of adjacent pixels having pixel values corresponding to a temperature range of approximately 32° C. to approximately 41° C. can be identified as a coarse sub-region. A given cluster having a generally circular or oval shape is identified as the coarse sub-region, the cluster being expected to correspond to the face of the person captured in the thermal image. The detection can further extend the coarse sub-region from the oval-shaped cluster so that the coarse sub-region covers the full body of the person. This extending can be based on an approximately body shape and nearby pixels having temperature values close in value to an expected temperature value of a human body. An extended cluster having pixel values corresponding to a temperature range of approximately 25° C. to approximately 42° C. can be identified as corresponding to the body of the person.

The respective coarse sub-region of each thermal image of the subset is compared with the coarse sub-region of the non-thermal image to determine a respective degree of matching. The comparison can evaluate the degree of matching between the pixel coordinates defining the non-thermal coarse sub-region against the pixel coordinates defining each respective thermal coarse sub-region. Where an adjustment/transformation to the pixel coordinates of the thermal images and/or the non-thermal image is applied to account for the positional/spatial offset between the positions and orientations of the non-thermal camera 108 and the thermal camera 116 (ex: affine transformation), the comparison can be carried out after applying the adjustment. The comparison is carried out to identify a given thermal image from the subset of thermal images for which its thermal coarse sub-region presents a sufficient correspondence with the coarse sub-region of the given non-thermal image (where appropriate, after applying the transformation of coordinates to account for positional/spatial offset of the cameras). The given thermal image having this sufficient correspondence is selected as the synchronized thermal image corresponding to the given non-thermal image.

According to one example embodiment, the synchronizing process can be repeated for each non-thermal image containing the person within the scene to identifying a corresponding synchronized thermal image.

According to another example embodiment, the synchronizing process is carried out once for each instance of a person detected within the scene. After synchronizing the series of non-thermal images with the series of thermal images by identifying a corresponding synchronized thermal image for the given non-thermal image, subsequent non-thermal images in the series and subsequent thermal images in the series are determined to correspond with each other based on this first synchronization.

According to one example embodiment, the non-thermal coarse region of the given non-thermal image is defined by a bounding box (where appropriate, after applying the transformation of coordinates to account for positional/spatial offset of the cameras). A thermal coarse region of a thermal image is determined to present a sufficient correspondence if the thermal coarse region sufficiently fits within the bounding box. Where the thermal coarse regions of multiple thermal images fit within the bounding box, the thermal image having a coarse sub-region presenting a best/maximum fit within the bounding box is selected as the corresponding synchronized thermal image for the given non-thermal image.

Continuing with FIG. 1, the region of interest detection module 124 is further configured to determine a distance value that is representative of a physical real-life distance between the person in the scene 148 and the thermal camera 116.

According to one example embodiment, the non-thermal camera 108 is positioned and oriented to capture the full body of the person, including the feet of the person. In particular, the contact point between the person's feet and the ground is captured.

The determining of the distance value (representative of the physical distance of the person from the thermal camera) includes detecting the feet sub-region within the body sub-region of the non-thermal image. For example, where a bounding box is defined to represent the body sub-region, the bottom of the bounding box can be defined as the feet sub-region. Alternatively, an additional feet detection algorithm can be applied to the captured non-thermal image to detect the feet sub-region of the person.

The distance value is further determined based on the position of the feet sub-region within the non-thermal image. The position of the feet sub-region can be defined by pixel coordinates corresponding to the feet sub-region within the non-thermal image.

During a calibration phase, distance values for each of a plurality of pixel positions within a calibration non-thermal image are pre-defined. For example, a set of distance lines can be drawn virtually within a non-thermal image frame and a respective distance value is also pre-defined for each of the distance lines. Then during operation, the distance value for a given captured person subject can be determined based on the position of the feet sub-region within the non-thermal image relative to the pre-defined virtual distance lines. The pre-defined virtual distance lines can be defined radially relative to the non-thermal camera 108.

The distance value determined from the non-thermal image can be further adjusted to account for any spatial offset between the non-thermal camera 108 and the thermal image camera 116. The adjusted distance value represents the distance value between the position of the person and the thermal camera 116.

The distance value can represent the distance over an x-axis, i.e. in a direction parallel to an underlying plane, i.e. the ground in the scene over which the person is traveling.

The distance value is used to normalize the measured temperature value that is previously determined based on the region of interest of the captured thermal image(s) (ex: corresponding to the forehead sub-region of the captured non-thermal image(s)). It will be understood that since the thermal camera 116 measures the level of black body radiation and the level of radiation decreases over distance, the amount of measured radiation will decrease (or increase) according to an increase (or decrease) in the distance at which the thermal camera 116 measures the radiation from an emission source. The normalization of the measured temperature value based on the distance value seeks to correct for such differences.

In a calibration phase, a black body radiation source having a known emitted temperature or a known range of emitted temperatures is moved over a distance sweep. Measurements are taken at the thermal camera 116 as the blackbody radiation source is moved over the distance sweep. The variance in the measurements made by the thermal camera 116 as the blackbody radiation source is moved is used to define a temperature/distance variation curve. Other calibration steps can also be applied, as described elsewhere herein.

In operation, the temperature/distance variation curve can be applied to determine the amount of adjustment to be applied to the measured temperature value based on the distance value determined.

According to various example embodiments, the temperature/distance variation curve can be defined over a distance range of approximately 2 metre to 7 metres.

According to various example embodiments, the thermal screening system 100 receives ambient temperature and/or humidity measurements, which may be received at the temperature determination module 140. The ambient temperature and/or humidity measurements are a measurement of the ambient temperature (i.e. room temperature) and/or humidity level measured within the physical space corresponding to the scene captured by the thermal camera. A measured temperature/ambient temperature-humidity curve can be defined over a range of ambient temperatures and humidity levels, and the measured temperature value can be adjusted according to this curve. Where the measured temperature value has already been normalized according to distance value, this normalized temperature is further adjusted based on ambient temperature and/or humidity level, thereby producing a further normalized temperature value.

According to various example embodiments, the temperature value (which may be normalized according to the distance value and/or the ambient temperature and/or humidity level) can be further normalized based on one or more characteristics of the person captured by the non-thermal camera 108 and the thermal camera 116. It was observed that due to differences in persons in different demographic groups including age ranges and gender groups, different skin temperature thresholds are used at which a given person is considered to indicate a presence of a possible abnormally elevated temperature.

According to one example embodiment, the region of interest detection module 124 is configured to determine a set of temperature-related characteristics related to the person based on visual information in the non-thermal image(s). As described hereabove, the region of interest detection module 124 detects a face sub-region of the person with the non-thermal image. A facial analysis algorithm can further be applied to the face sub-region of the person to predict the one or more characteristics related to the person gender group or age range. The one or more characteristics may include one or more of a predicted age range and a gender group of the person within the scene.

In operation, the temperature value (which may be normalized according to the distance value and/or the ambient temperature and/or humidity level) is further adjusted based on the temperature-related characteristics determined for the person subject, thereby producing a further normalized temperature value.

The determination of the temperature-related characteristics using the facial analysis algorithm is carried out in a privacy-aware manner (i.e. processing privacy-sensitive information in a manner that particularly considers privacy-related issues, such as to ensure compliance with applicable privacy-related legislation, protocols or regulations (ex: General Data Protection Regulation (GDPR)). The processing of the non-thermal image(s) to determine the predicted temperature-related characteristics can be carried out in accordance with privacy-aware methods described in U.S. provisional applications Nos. 62/970,482 and 63/085,515, which are incorporated herein by reference.

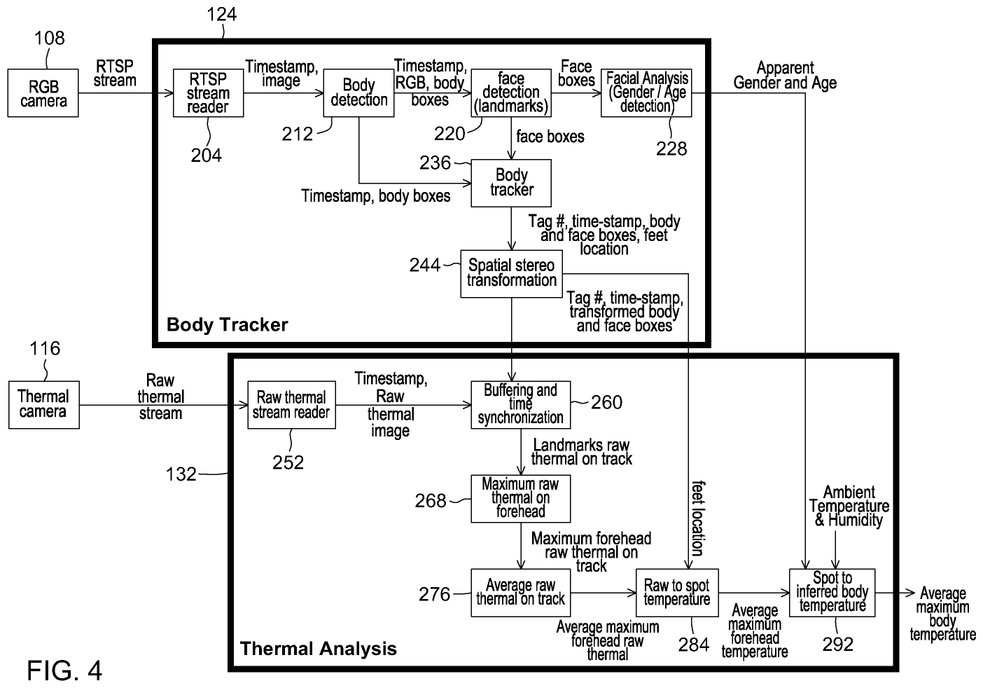

Referring now to FIG. 4, therein illustrated is a schematic diagram of detailed modules and operations within the thermal screening system according to one example embodiment.

The non-thermal (ex: RGB) camera 108 captures a stream of images of the scene.

An image stream reader 204, which may be part of the region of detection module 124, processes the stream of images to output discrete non-thermal images that are each time-stamped.

A body detection module 212, which is part of the region of interest detection module 124, processes each image of the stream of non-thermal images and detects the body sub-region for each image. A time-stamped bounding box 180 is outputted.

A face detection module 220, which is part of the region of interest detection module 124, processes the portion of each non-thermal image outlined by the body bounding box 180 and further detects the face sub-region. The face detection module 220 further processes the face sub-region to determine facial landmarks (ex: eyes, nose, mouth).

The face sub-region of at least one non-thermal image is received at a face categorization module 228, which applies facial analysis thereto to determine temperature-related characteristics of the captured person subject. The characteristics can be a gender group and/or age range of the captured person subject. The characteristics of the captured person subject are outputted by the facial analysis module 228.

A body tracking module 236, which is part of the region of interest detection module 124, can co-operate with the body detection module 212, to track the position of the body sub-region across the plurality of captured non-thermal images. The feet sub-region can be further detected in each body sub-region. A series of bounding boxes, each corresponding to a respective captured non-thermal image, is outputted from the body tracker. Each bounding box can be tagged with a unique ID, time-stamped and further indicate the location of the face sub-region and feet sub-region therein.

A spatial transformation module 244 determines, for the sub-region of each captured non-thermal image, a distance value for that non-thermal image.

The thermal (ex: infrared) camera 116 captures a stream of images of the scene.

An image stream reader 252, which may be part of the synchronization module 132, processes the stream of images to output discrete thermal images that are each time-stamped.

A buffering time synchronization module 260 receives the timestamped discrete thermal images, temporarily stores (buffers) the images, and identifies, for each discrete non-thermal image, a corresponding thermal image. The synchronization module 260 further identifies the corresponding region of interest for each thermal image based on the received coordinates of the region of interest of the corresponding non-thermal image.

A temperature determination module 268 determines for the region of interest of each thermal image a measured temperature value. The temperature value can be determined from the maximum pixel values within the region of interest of each thermal image. As described elsewhere herein, a series of measured intermediate temperature values are determined.

An average temperature module 276 calculates an average measured temperature by taking an average of the series of intermediate temperature values.

A first normalizing module 284 receives the distance value from the spatial transformation module 244, receives the average measured temperature from the average temperature module 276, and calculates a first normalized temperature value by making an adjustment to the average measured temperature according to the distance value. This first normalized temperature represents a spot temperature of the person.

A second normalizing module 292 receives the first normalized temperature value, receives the predicted temperature-related characteristics (ex: age range and/or gender group) of the person subject from the facial analysis module 228, receives an ambient temperature and humidity level, and calculates a second normalized temperature by making an adjustment to the first normalized temperature according to the predicted temperature-related characteristics, the ambient temperature, and the humidity level.

The second normalizing module 292 outputs an effective temperature value that is representative of the core body temperature of the person subject. This effective temperature value can be used for making a decision on a further thermal screening action.

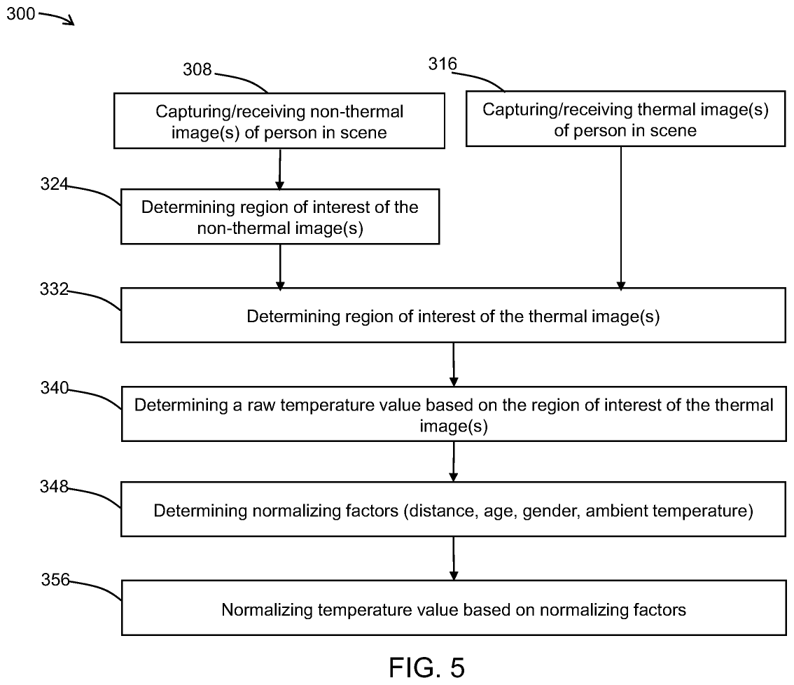

Referring now to FIG. 5, therein illustrated is a flowchart showing the operational steps of a method 300 for performing thermal screening according to one example embodiment. The method 300 can be generally carried out at the thermal screening system 100 as described herein according to various example embodiments. In other embodiments, a non-transitory computer-readable medium can be provided with instructions stored thereon which, when executed, perform the method 300.

At step 308, non-thermal image(s) captured by a non-thermal camera 108 are received. The non-thermal image(s) can be received at the region of interest determination module 124.

At step 316, thermal image(s) captured by a thermal camera 116 are received. The thermal image(s) can be received at the camera synchronization module 132.

At step 324, the region of interest of each of the non-thermal image(s) is determined. Step 324 can be carried out by the region of interest determination module 124.

At step 332, the region of interest of the thermal image(s) is determined. Step 332 can include matching a non-thermal image with its corresponding thermal image. Step 332 can be carried out by the camera synchronization module 132.

At 340, a raw measured temperature value is determined based on the region of interest of the thermal image(s). Step 340 can be carried out by the temperature determination module 140.

At step 340, one or more normalizing factors, such as distance value, person's age range, person's gender group, and ambient temperature and/or humidity are determined. It will be understood that these normalizing factors can be determined at different phases of method 300 and can be carried out prior to step 340.

At step 356, the raw measured temperature value is normalized based on the normalizing factors determined at step 348. Step 356 can be carried out by the temperature determination module 140. A normalized temperature value representative of the core temperature of the person captured in the non-thermal and thermal images is outputted.

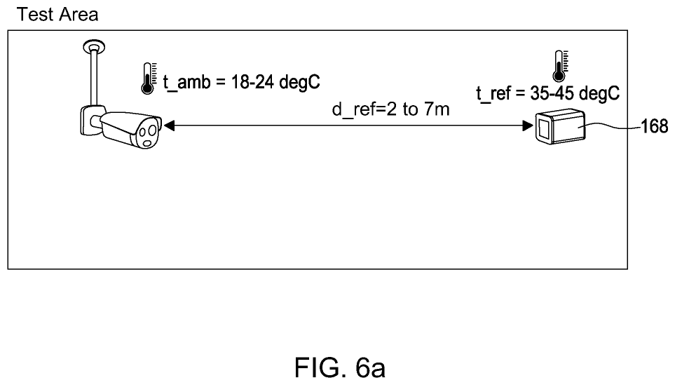

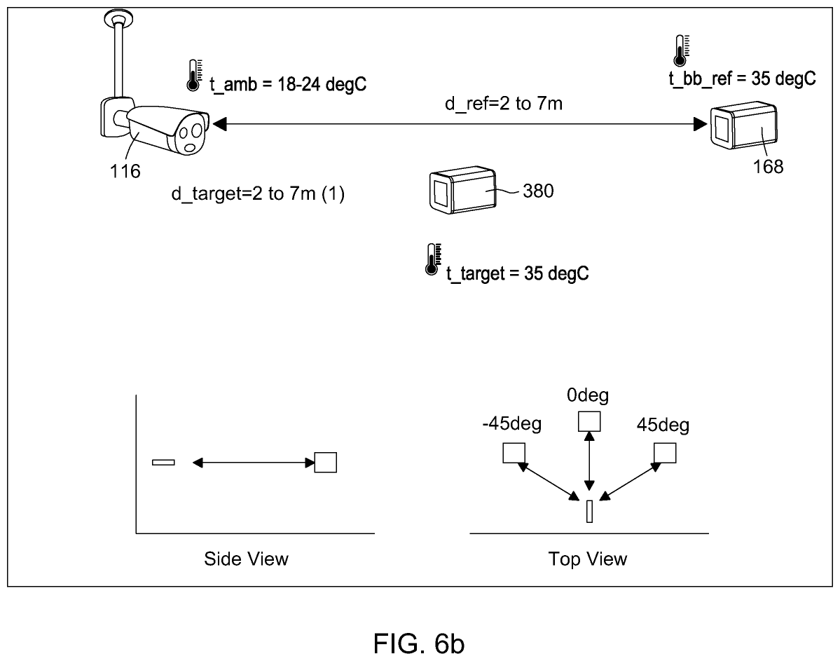

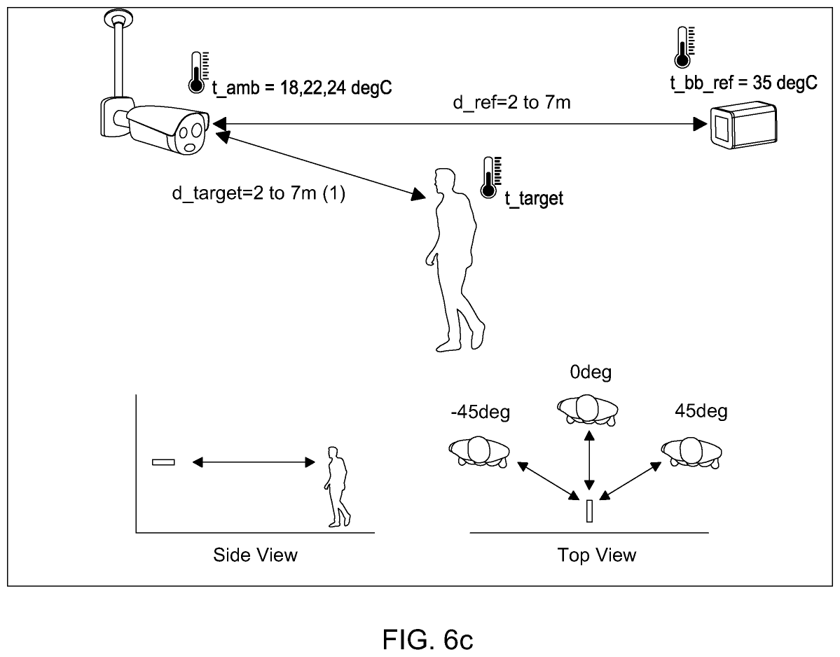

Referring now to FIG. 6a, therein illustrated is a schematic diagram of first calibration phase of an experimental setup according to one example embodiment. The experimental setup was implemented to validate the thermal screening described herein. In the first calibration phase, the thermal camera 116 is calibrated for a first set of parameters. The parameters are varied over three dimensions, being a distance of the reference blackbody emitter 168 over a distance range of 2 to 7 metres, the temperature outputted by the reference blackbody emitter 168 over a temperature range of 35 to 45 degrees Celsius (in 0.5 degrees Celsius steps), and the ambient temperature over a temperature range of 18 to 24 degrees Celsius. Readings of the reference blackbody emitter 168 are taken at various data points within the three dimensions of operating parameters. This first calibration phase seeks to calibrate the thermal camera 116 for different operating conditions, namely the distance of the reference blackbody emitter from the thermal camera, the ambient temperature, and the temperature outputted by the reference blackbody emitter.

Referring now to FIG. 6b, therein illustrated is a schematic diagram of a second calibration phase of the experimental setup according to the example embodiment. In the second calibration phase, a second blackbody emitter 380 is provided to act as a test subject. The reference blackbody emitter 168 is set at an operating reference temperature falling within the range defined in the first calibration phase. Measurements of the second blackbody emitter 380 are taken by the thermal camera 116 as it is moved over a distance range (ex: distance of 2 to 7 metres). The variance in the measured temperature is recorded as the distance is varied. This variance is used to define the temperature/distance curve used to provide the first normalization of the raw measured temperature in operation. Measurements of the second blackbody emitter 380 are taken as the angle of the second blackbody emitter 380 relatives to a central capture axis of the thermal camera 116 is varied. These measures can be used to define a second normalization curve. Measurements of the second blackbody emitter 380 can also be taken as other parameters are varied, such as relative humidity and ambient temperature, to define additional normalization curves. Accordingly, normalization over a plurality of dimensions is possible.

FIG. 6c illustrates a schematic diagram representing an operational setup. It will be appreciated that as compared to FIG. 6b, a person subject is shown in FIG. 6c instead of the second blackbody emitter 380. After determining the raw temperature of the region of interest of the person and the distance of the person to the thermal camera 116, a normalized temperature can be determined based on the adjustment curves determined in the second calibration phase and based on reference calibration parameters determined in the first calibration phase.

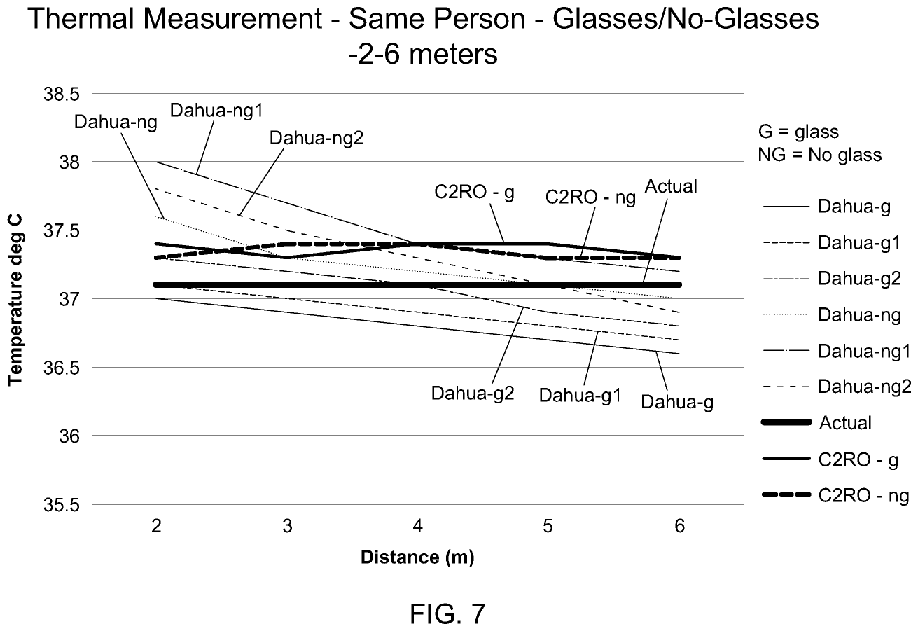

FIG. 7 is a graph showing the performance of the thermal screening according to the experimental setup described herein in comparison to a commercially available thermal screening system. The measurements are made for a single person in the same way as the experimental setup described with reference to FIGS. 6a to 6c. There are two sets of variables for this experiment while the person, the room and its ambient condition including the temperature, and reference blackbodies are identical:
the distance from 2 to 7 meters
the person with glasses (X-g) and the same person with no glasses (X-ng)

It will be appreciated that measurements taken for a commercially available solution (Dahua) show obtained temperature values with a significant non-flat slope over the distance range. This represents a significant variance in the obtained temperature value, despite the same person being measured with glasses (Dahua-g) or with no glasses (Dahua-ng) with the ambient condition.

Results obtained using the experimental setup according to examples described herein with the person wearing glasses or no glasses (respectively named C2RO-g, C2RO-ng) exhibit a substantially flat temperature value slope over the distance range, reflecting consistent obtained temperature value over the distance range.

The experimental setup achieved an accuracy of about approximately 0.15 degrees Celsius (measured temperature versus using the experimental setup versus a real temperature). This level of accuracy would allow for automated passive thermal screening with a significantly reduced need for regular calibration.

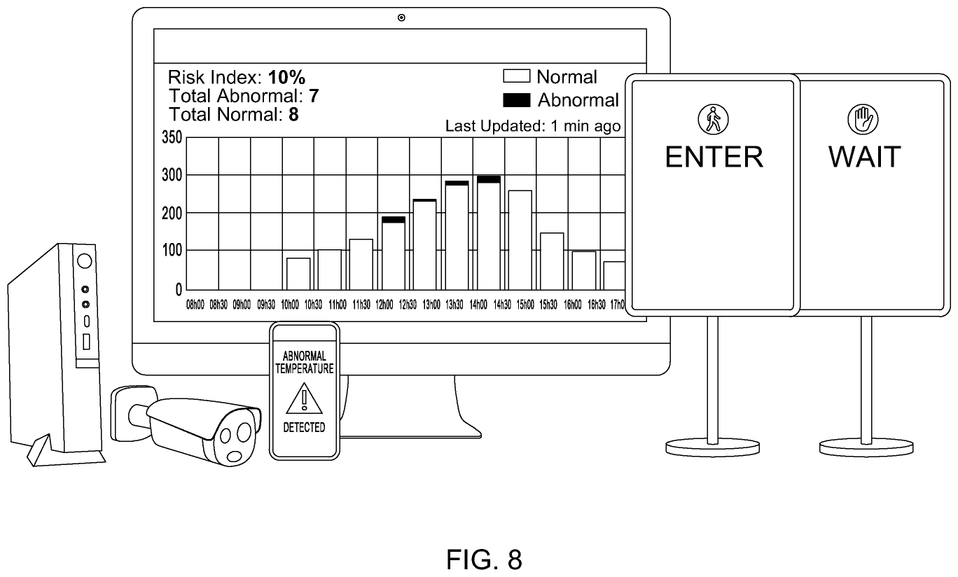

FIG. 8 shows hardware components of an automated thermal screening system according to one example embodiment. While the edge server and the camera are the processing and sensor components respectively, the phone, the screen, and the digital signage are sample devices to visualize the data and alerts.

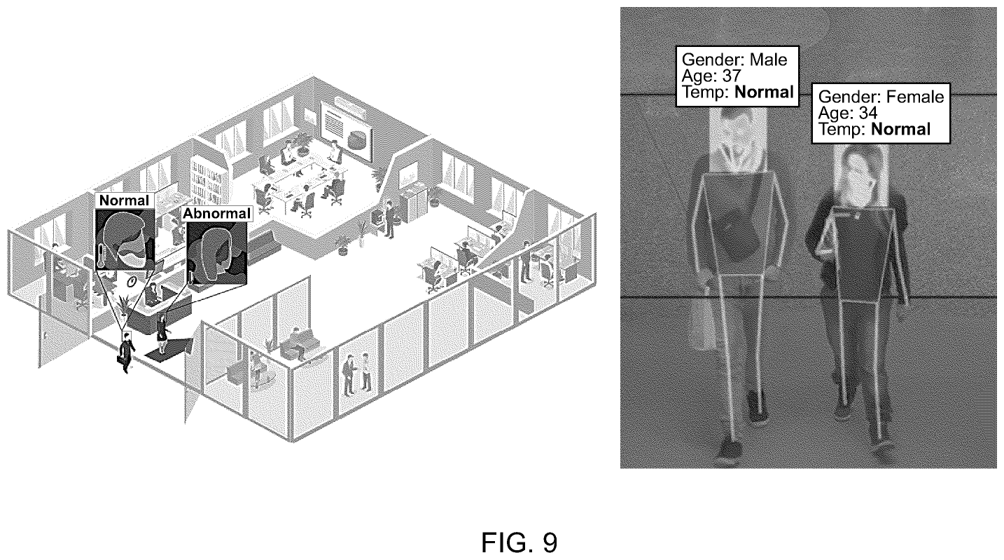

FIG. 9 illustrates a use case for the thermal screening system according to one example embodiment.

It will be appreciated that various exemplary embodiments for thermal screening described herein allow for performing thermal screening with greater accuracy and in a more discreet manner. Advantageously, by normalizing the measured temperature value based on distance and ambient temperature/humidity, the thermal screening can be implemented with less strict operating conditions. For example, by being able to be operated in a range of ambient temperatures and humidity levels, a less strict over the ambient environment is required, or no need for frequent calibration and tunning.

By being able to be operated over a distance range, a person subject does not have to be strictly positioned at a precise distance relative to the capturing thermal camera. In some implementations, the person subject does not need to adapt their normal behavior while the thermal screening is actually being implemented. That is, the person can traverse a space being monitored by the thermal screening system as if the system is not present, i.e. passive thermal screening. As the person walks through space, such as through a door entrance, a plurality of images (thermal and non-thermal) is captured and the person is tracked across the images. Temperature value can be determined for the person while accounting for the change in distance as the person is walking. The thermal screening can be carried out without the person being aware that the screening is carried out or need to do any action including taking out the glasses or hats or other accessories.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A method for performing automated thermal screening, the method comprising:
capturing at least one non-thermal image of a scene having a person within the scene;
capturing at least one thermal image of the scene having the person within the scene;
determining a region of interest of the non-thermal image;
determining, based on a body sub-region of the non-thermal image corresponding to the body of the person within the scene, a distance value representative of a physical distance of the person from the thermal camera;
determining a region of interest of the thermal image corresponding to the region of interest of the non-thermal image;
determining a raw temperature value for the person based on the region of interest of the thermal image; and
normalizing the raw temperature value based on at least the distance value to determine a normalized temperature value.

2. The method of claim 1, wherein the non-thermal image of the scene includes capturing of a full body of the person within the scene;
wherein the body sub-region of the non-thermal image corresponds to the full body of the person within the scene.

3. The method of claim 2, wherein determining the distance value comprises detecting a feet sub-region within the body sub-region of the non-thermal image; and
wherein the distance value is determined based on the position of the feet sub-region within the non-thermal image.

4. The method of claim 1, wherein determining the region of interest of the non-thermal image comprises:
determining, within the body sub-region, a face sub-region corresponding to the face of the person within the scene.

5. The method of claim 4, wherein determining the region of interest of the non-thermal image further comprises:
determining, within the face sub-region, one or more facial landmarks.

6. The method of claim 5, wherein the one or more facial landmarks comprise one or more of eyes landmarks, a nose landmark, corners of the mouth landmarks.

7. The method of claim 5, wherein the region of interest of the thermal image is determined relative to one or more positions of the facial landmarks of the non-thermal image.

8. The method of claim 7, wherein the region of interest of the thermal image corresponds to the forehead of the person.

9. The method of claim 1, wherein capturing the at least one non-thermal image comprises capturing a series of a plurality of non-thermal images of the scene having the person within the scene;
wherein capturing at least one thermal image comprises capturing a series of a plurality of thermal images of the scene having the person within the scene; and
wherein the region of interest is determined for each of the plurality of non-thermal images to track the region of interest over the series of the plurality of non-thermal images;
wherein a respective region of interest is identified for each of the plurality of thermal images based on the region of interest of a corresponding non-thermal image; and
wherein a respective temperature value is determined based on the region of interest of each of the thermal images.

10. The method of claim 9, wherein an average temperature value is calculated from an average of the temperature values determined based on the region of interest of each of the thermal images.

11. The method of claim 9, further comprising synchronizing the series of non-thermal images with the series of thermal images, the synchronizing comprising:
for a given non-thermal image of the series of non-thermal images, detecting, a non-thermal coarse sub-region corresponding to the person within the scene;
for the given non-thermal image, identifying a subset of the thermal images taken within a temporal neighborhood of the given non-thermal image;
for each thermal image of the subset, detecting a respective thermal coarse sub-region corresponding to the person within the scene;
identifying, from the subset of the thermal images, a given thermal image having its thermal coarse sub-region presenting a sufficient correspondence with the coarse sub-region of given non-thermal image, the given thermal image being selected as the synchronized thermal image corresponding to the given non-thermal image.

12. The method of claim 11, wherein the non-thermal coarse region is defined by a bounding box; and
wherein the sufficient correspondence is present when the thermal coarse sub-region of the given thermal image sufficiently positionally fits within the bounding box.

13. The method of claim 12, wherein the thermal image of the subset of thermal images having a coarse sub-region presenting a maximum fit within the bounding box is selected as the corresponding thermal image for the given non-thermal image.

14. The method of claim 11, wherein the non-thermal coarse sub-region corresponds to the body sub-region of the non-thermal image; and
wherein the thermal coarse sub-region of each thermal image corresponds to a body sub-region of each thermal image corresponding to the body of the person within the scene.

15. The method of claim 1, wherein the normalized temperature value is further determined based on an ambient temperature in a physical space corresponding to the captured scene.

16. The method of claim 1, further comprising:
detecting a face sub-region corresponding to a face of the person within the scene;
applying facial analysis to determine a predicted age range and/or gender group of the person within the scene;
wherein the normalized temperature value is further determined based on the predicted age range and/or gender group of the person.

17. The method of claim 1, wherein the at least one non-thermal image is captured by a non-thermal camera and the at least one thermal image is captured by a thermal camera;
wherein the non-thermal camera and the thermal camera are integrated within a hybrid camera system.

18. The method of claim 1, wherein the at least one non-thermal image is captured by a non-thermal camera and the at least one thermal image is captured by a thermal camera;
wherein the non-thermal camera and the thermal camera are separate devices;
wherein the non-thermal camera is positioned relative to the thermal image by a known positional offset; and
wherein the region of interest of the thermal image corresponding to the region of interest of the non-thermal image is determined based on the known positional offset.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A system for performing automated thermal screening, the system comprising:
at least one processor;
at least one memory coupled to the processor and storing instructions executable by the processor and that such execution causes the processor to perform operations comprising:
capturing at least one non-thermal image of a scene having a person within the scene;
capturing at least one thermal image of the scene having the person within the scene;
determining a region of interest of the non-thermal image;
determining, based on a body sub-region of the non-thermal image corresponding to the body of the person within the scene, a distance value representative of a physical distance of the person from the thermal camera;
determining a region of interest of the thermal image corresponding to the region of interest of the non-thermal image;
determining a raw temperature value for the person based on the region of interest of the thermal image; and
normalizing the raw temperature value based on at least the distance value to determine a normalized temperature value.

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. A non-transitory computer-readable medium having instructions stored thereon which, when executed by one or more processors of a computing system, cause the computing system to carry out a method for performing automated thermal screening, comprising:
capturing at least one non-thermal image of a scene having a person within the scene;
capturing at least one thermal image of the scene having the person within the scene;
determining a region of interest of the non-thermal image;
determining, based on a body sub-region of the non-thermal image corresponding to the body of the person within the scene, a distance value representative of a physical distance of the person from the thermal camera;
determining a region of interest of the thermal image corresponding to the region of interest of the non-thermal image;
determining a raw temperature value for the person based on the region of interest of the thermal image; and
normalizing the raw temperature value based on at least the distance value to determine a normalized temperature value.
